(19) United States
(12) Patent Application Publication
(10) Pub. No.: US 2026/0224853 A1
(43) Pub. Date: Aug. 6, 2026

(54) SYSTEMS, DEVICES, APPARATUSES AND METHODS FOR RETAINING ELONATED STRUCTURES WITHIN TISSUE

(71) Applicants: Sanjay SINHA, Avon, CT (US); Gate Science, Inc., Moultonborough, NH (US)

(72) Inventor: Sanjay Sinha, Avon, CT (US)

(21) Appl. No.: 19/152,427

(22) PCT Filed: Jan. 30, 2024
(86) PCT No.: PCT/US2024/013542

§ 371 Date: Jul. 30, 2025

Related U.S. Application Data
(60) Provisional application No. 63/442,100, filed on Jan. 30, 2023.

(51) Int. Cl. A61M 25/04; A61M 25/01; A61N 1/05; A61N 1/36

(52) U.S. Cl. CPC A61M 25/04; A61M 25/0105; A61N 1/05; A61N 1/36071; A61M 2025/0166

(57) ABSTRACT

Some embodiments of the present disclosure provide for an anchoring apparatus for reversibly retaining at least one of catheter and a needle (for example or other associated device) within tissue. The apparatus includes a cylinder including a wall, an outer surface, and one or more tines. In some embodiments, each tine includes a portion thereof being at least one of flexibly integral and flexibly affixed to at least one of the outer surface and the wall and can be configured to project away from the surface of the cylinder when in a deployed state. In a undeployed state, each tine can be configured to wrap or otherwise fold upon rotation of the cylinder in a first direction so as to at least one of lie immediately adjacent the outer surface and be received by a recess or opening in at least one of the surface of the cylinder and wall of the cylinder.

### RELATED APPLICATIONS

This application claims benefit of and priority to U.S. provisional application no. 63/442,100, filed Jan. 30, 2023, and entitled, “SYSTEM, DEVICE AND METHOD FOR RETAIING TUBE WITHIN TISSUE. Some embodiments of inventions of the present disclosure are also related to PCT publication no. WO2022221452A1. Both of the forgoing disclosures are herein incorporated by reference in their entireties.

### BACKGROUND

Elongated structures, including tubes, catheters, leads, and/or other solid and lumen-based structures (hereinafter throughout this disclosure together referred individually to as “catheter”, and a plurality thereafter as “catheters”, regardless of whether such structure includes a lumen or not, i.e., solid structures) and various other instruments are often used in numerous medical procedures to deliver and/or remove fluid, air, electrical pulses/current, radio-frequency, implants, and the like, to tissue. Such structures traverse tissue, and more particularly, the skin. Often times precise placement of the structure is required, both with respect to the skin or tissue of initial placement as well as the end of a structure, as to keep the structure precisely placed during use. In certain cases, for example, linear movement of the structure (e.g., along its longitudinal axis) is detrimental to the associated medical procedure being formed.

Accordingly, there is a need to retain or at least substantially retain movement of a structure within tissue in position so that an associated medical procedure being performed.

### SUMMARY OF THE DISCLOSURE

Embodiments of inventions of the present disclosure are directed at least to an anchoring apparatus for reversibly retaining a device in tissue, and more particularly, reversibly retaining at least one of tube, a catheter and a needle within tissue.

In some embodiments, an anchoring apparatus for reversibly retaining a catheter or tube within tissue is provided. The apparatus includes a cylinder including a wall, an outer surface, and one or more tines. Each tine includes a portion thereof being at least one of flexibly integral and flexibly affixed to at least one of the outer surface and the wall and is configured to project away from the surface of the cylinder when in a deployed state. In a undeployed state, is configured to wrap or otherwise fold upon rotation of the cylinder in a first direction so as to at least one of lie immediately adjacent the outer surface and/or be received by a recess or opening in at least one of the surface of the cylinder and wall of the cylinder.

Some variations of at least the above-noted embodiment may further include one and/or another, and in some variations, a plurality of, and in some variations (if not mutually exclusive), a majority of, and/or substantially all of (and still further, in some variations, all of) the following elements, features, functionality, structure, materials, steps, and/or clarifications, yielding yet further embodiments of the disclosure:
the one or more tines comprise a plurality of tines;
the plurality of tines are arranged around the circumference of the cylinder;
the plurality of tines are equally spaced around the circumference of the cylinder;
each tine is configured to be arranged within tissue;
each tine includes a surface area configured to aid in retaining at least one of a catheter and needle within tissue, optionally including a structural rigidity to retain the tine in place within tissue so as not to fold or collapse under use;
each tine includes at least one of a height, relative to the surface of the cylinder and an edge configured to interact with tissue so as to keep at least a distal end of a/the catheter within the tissue upon an axial force being placed on the catheter or a component in a proximal direction thereof;
each tine includes a barb portion configured to interact with tissue so as to keep the catheter within the tissue and prevent longitudinal movement of the catheter at least in a first direction;
each tine is configured as a propeller-like structure in the deployed state;
the propeller-like structure is configured with a pitch between 1 and 90 degrees relative to an axis of the cylinder;
the one or more tines or plurality of tines comprise at least two tines;
the one or more tines or plurality of tines comprise at least three tines;
the one or more tines or plurality of tines comprise at least four tines;
the one or more tines or plurality of tines are arranged in one or more planes lying at an angle to an/the longitudinal axis of the cylinder, wherein optionally, the angle is perpendicular the longitudinal axis;
the one or more tines or plurality of tines are arranged in one or more planes arranged at an/the longitudinal axis of the cylinder;
the one or more tines or plurality of tines are arranged in at least two planes arranged at an angle to the longitudinal axis of the cylinder;
the catheter includes one or more electrodes;
the catheter includes or is associated with a pulse generator;
the one or more tines or plurality of tines are arranged in at least two planes lying at an angle to an/the longitudinal axis of the cylinder, wherein optionally, the angle is perpendicular the longitudinal axis;
the one or more tines or plurality of tines are arranged in at least two planes lying at an angle to an/the longitudinal axis of the cylinder,
optionally, the angle is perpendicular the longitudinal axis;
a/the barb associated with the one or more or plurality of tines arranged on a first of the at least two planes is/are shaped and arranged so as to prevent longitudinal movement of the catheter in a first direction;
and
a/the barb associated with the one or more or plurality of tines arranged on a second of the at least two planes is/are shaped and arranged so as to prevent longitudinal movement of the catheter in a second direction opposite to the first direction.

In some embodiments, a system for reversibly retaining a catheter within tissue is provided and includes the apparatus of any of the embodiments disclosed in this disclosure, including those set out above, and at least one of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and only one of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and at least two of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and only two of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and at least three of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and only three of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and at least four of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and only four of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and at least five of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and only five of: a catheter, a needle, an electrode, a liner extrusion, a jacket extrusion and a conductor wire.

In some embodiments, a system for reversibly retaining a catheter within tissue is provided and includes the apparatus of any of the embodiments disclosed in this disclosure, including those set out above, and a catheter, a needle, an electrode, a liner extrusion, a jacket extrusion and a conductor wire.

In some embodiments, a method for retaining at least a catheter within tissue is provided and includes providing the anchoring apparatus or system according to any of such embodiments disclosed herein including those set out above, where the catheter and cylinder form an assembly, inserting the assembly through the surface of the insertion site towards a target area, where rotating the cylinder in a first direction such that the one or more tines of the anchoring apparatus deploy within the tissue. The method further includes rotating the cylinder in a second direction opposite to the first direction such that the one or more tines of the anchoring apparatus wrap and/or otherwise fold to lie immediately adjacent the outer surface of the cylinder and optionally are received by a recess or opening in at least one of the surface of the cylinder and wall of the cylinder.

In some embodiments, a method for retaining at least a catheter within tissue is provided and includes providing the anchoring apparatus or system according to any of such embodiments disclosed herein including those set out above, where the catheter and cylinder form an assembly and the catheter includes one or more electrodes to apply an electric current to tissue. The method can include any one or more, and preferably, a plurality of, and in some embodiments, a majority of, and in some embodiments, substantially all of, and in some embodiments, all of the following (leading to yet further embodiments of the present disclosure):
sterilizing a surface of tissue at an insertion site;
anesthetizing the surface at the insertion site with a local anesthetic;
inserting the assembly through the surface of the insertion site towards a target nerve optionally under ultrasound imaging guidance, where at least one of ultrasound images produced by the ultrasound imaging guidance and elicitation of an appropriate muscle twitch of a muscle proximate the target nerve via the one or more electrodes is configured to confirm that a portion of catheter is adjacent the target nerve;
rotating the cylinder in a first direction such that the one or more tines of the anchoring apparatus deploy within the tissue;
and
rotating the cylinder in a second direction opposite to the first direction such that the one or more tines of the anchoring apparatus wrap and/or otherwise fold to lie immediately adjacent the outer surface of the cylinder and optionally are received by a recess or opening in at least one of the surface of the cylinder and wall of the cylinder.

In some embodiments, a method for use of a catheter pain management apparatus or system is provided and includes providing the anchoring apparatus or system according to any of such embodiments disclosed herein including those set out above, the catheter and cylinder (and, in some embodiments, optionally a needle), form an assembly and the catheter includes a portion including a medical device for positioning and/or delivering the medical device adjacent a particular area of tissue, optionally removably affixing a/the needle (if present) to the catheter, connecting the needle to a pulse generator configured to deliver a pulses of current at a predetermined amperage, frequency, and/or duration, connecting tubing configured to be in continuity with a lumen of the needle to a syringe filled with a dextrose solution, flushing the lumen of the tubing and needle so as to remove any air therefrom, sterilizing a surface of tissue at an insertion site, anesthetizing the surface at the insertion site with a local anesthetic, injecting a dextrose solution through the needle to create an areas of fluid by hydro-dissection adjacent to the target nerve, and inserting the assembly through the surface of the insertion site towards a target nerve optionally under ultrasound imaging guidance. At least one of ultrasound images produced by the ultrasound imaging guidance and elicitation of an appropriate muscle twitch of a muscle proximate the target nerve via the one or more electrodes is configured to confirm that the tip of the needle is adjacent the target nerve. In some embodiments, the method can include one or more of the following, and in some embodiments, a plurality of, and in some embodiments, a majority of, and in some embodiments, substantially all of, and in some embodiment all of: un-affixing the needle from the catheter, sliding the catheter a predetermined distance into the hydro-dissected area adjacent the target nerve, removing the needle from the tissue, activating the pulse generator, optionally data linking the pulse generator to a smart device, where the smart device is configured to control the pulse generator via at least one of a dedicated application operating on the smart device and a user interface thereof, establishing at least one of a lower and upper stimulation threshold current for the pulse generator using at least one of the dedicated application and user interface, rotating the cylinder in a first direction such that the one or more tines of the anchoring apparatus deploy within the tissue so as to retain at least the catheter within the tissue, sealing the surface of the insertion site with a surgical glue, applying a waterproof dressing configured to cover the catheter outside the surface of the tissue so as to keep an injection port/hub thereof outside the dressing, closing the injection port/hub with a clave, injecting a local anesthetic solution through a syringe to create a primary nerve block, capping the clave with a luer-lock component, and to remove the catheter, rotating the cylinder in a second direction opposite to the first direction such that the one or more tines of the anchoring apparatus wrap or otherwise fold to lie immediately adjacent the outer surface of the cylinder and optionally received by a recess or opening in at least one of the surface of the cylinder and wall of the cylinder.

These and other features, functions/functionality, steps, structures, and/or clarifications can be found in the following detailed description and corresponding figures, a brief description of which follows.

### BRIEF DESCRIPTION OF THE DRAWINGS

FIG. 1A is a side view of an anchoring apparatus according to some embodiments of the disclosure;

FIG. 1B is an end view of the anchoring apparatus of FIG. 1A, according to some embodiments of the disclosure;

FIG. 1C is a cross-sectional view of the anchoring apparatus of FIG. 1A, according to some embodiments of the disclosure;

FIG. 1D is a perspective view of the anchoring apparatus of FIG. 1A, according to some embodiments of the disclosure;

FIG. 2 is a side view of an anchoring apparatus, according to some embodiments, used in association with a medical system/catheter for delivery electrical pulses to tissue, according to some embodiments.

### DETAILED DESCRIPTION

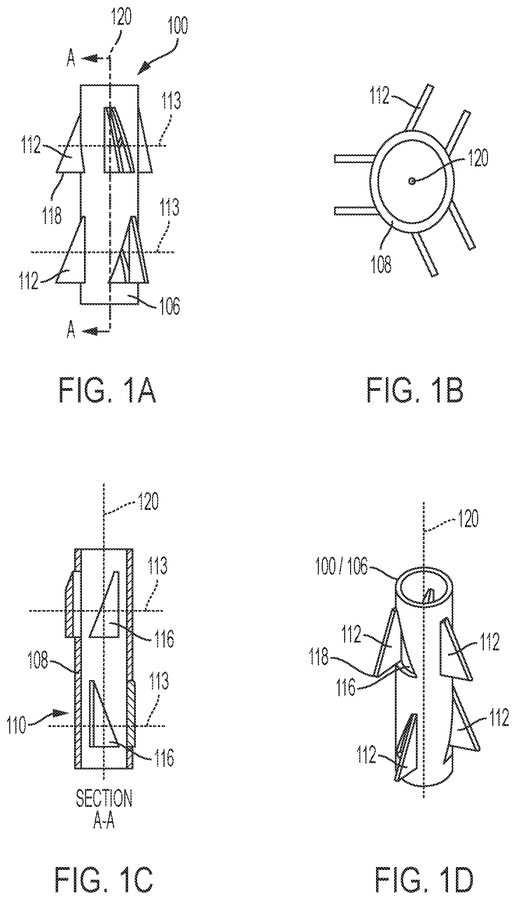

As shown in FIGS. 1A-D, an anchoring apparatus 100 for reversibly retaining at least one of catheter and a needle (for example) within tissue is provided. The apparatus 100 includes a cylinder or cylinder like structure 106, including a wall 108, an outer surface 110, and one or more tines 112. Each tine 112 can includes at least a portion thereof being at least one of flexibly integral and flexibly affixed to at least one of the outer surface 110 and the wall 108 and can be configured to project away from the surface 110 of the cylinder 106 when in a deployed state. In a undeployed state, each tine 112 can be configured to wrap or otherwise fold upon rotation of the cylinder 106 in a first direction 114 so as to at least one of each tine lies immediately adjacent the outer surface 110. In some embodiments, one or more tines 112 (preferably all tines), upon folding/wrapping, is received by a recess or opening 116 in at least one of the surface 110 of the cylinder 106 and wall 108 of the cylinder 106.

As shown in the figures, in some embodiments, a plurality of tines 112 is provided and can be arranged around a circumference of the cylinder 106 and can be equally spaced around the circumference of the cylinder 106.

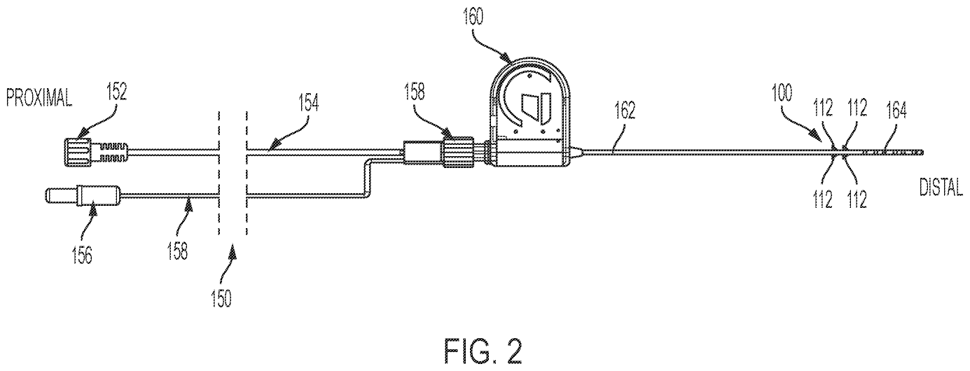

FIG. 2, which illustrates an example of a catheter 154, 162 of pain management device 150, configured for delivering at least one of electrical pulses and fluid (e.g., a medicament) for which the anchoring apparatus 100 can be used/affixed thereto. The catheter 154, 162 (embodiments of similar pain management devices are also disclosed in WO2022221452A1 incorporated herein by reference) can include a proximal connector 152 (which can be connected to a fluid source), a second connector 156 and associated tube 158 (which can correspond to an electrical connector and wire/lead, respectively, for connecting an external electrical stimulator to device 150), and a third connector 158, which can be a luer-lock. A pulse generator 160 is connected to the catheter/tube 154 and/or 158 via the luer-lock, as well as a distal tube/catheter 162 for delivering fluid and/or electrical stimulation (e.g., via electrodes 164) to tissue for which 100 the device 150 is inserted. In some embodiments, the anchoring apparatus 100 is affixed to a portion of the distal catheter 162 which is inserted within tissue to keep at least distal catheter 162 in position. The anchoring apparatuses 100 of the subject disclosure can be affixed at a point along the distal catheter thereof, according to some embodiments. Specifically, the anchoring apparatus can be affixed to portion of the catheter which is arranged within tissue at any point therein.

The surface area of each tine is configured to be between approximately 0.1 and 100 mm², which has been found to aid in retaining at least one of a catheter (and, in some embodiments, a needle) within tissue. Similarly, each tine can include at least one of a height between 0.5 mm and 10 mm, relative to the surface 110 of the cylinder 106 and an edge configured to interact with tissue so as to keep at least a distal end of the catheter 102 and/or needle 104 within the tissue upon an axial force being placed on the catheter 102 and/or needle 104 in a proximal direction thereof. Each tine, in some embodiments can include a geometric shape configured to perform the task of affixing the catheter in place; for example, at least one of a triangular shape (as illustrated), a curved shape, a “W” shape, an “M” shape, and a hook shape (e.g., “fishhook”). Such shapes include one more sharp or barb portions which can temporarily at least slightly embed in tissue to hold the catheter in place for a period of time necessary for performance of a medical procedure or aid.

In some embodiments, and as initially note above, one or more of the tines is configured as or includes at least one barb 118 portion configured to interact with tissue so as to keep the catheter and/or needle within the tissue. That said, in some embodiments, each tine 112 can be configured as or include a smooth portion or curved like structure configured to interact with tissue so as to keep the catheter 102 and/or needle 104 within the tissue. The geometry of the tine with a smooth edge provides an unexpected result of retention sufficient retention power to keep the catheter in place within the tissue The tines can have different geometric shapes can include semi-circular, semi-oval, triangular, square, rectangular and or polygonal with different number of sides.

In some embodiments, one or more tines 112 can be configured as a propeller-like structure in the deployed state (as shown), where the propeller-like structure can be configured with a pitch between 1 degree and 90 degrees relative to an axis of the cylinder 106 (and any and all ranges therebetween).

The one or more tines can include between 1-1000 (or more), and preferably less than 100, and more preferably less than 50, and most preferably between 1-20, 1-19, 1-18, 1-17, 1-16,1-15, 1-14, 1-13, 1-12, 1-11, 1-10, 1-9, 1-8, 1-7, 1-6, 1-5, 1-4, 1-3, 1-2, 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19 and 19-20.

A plurality of tines 112 can be arranged in one or more planes 113 lying perpendicular to an axis 120 of the cylinder 106. The one or more planes can be preferably less than 10 planes, and most preferably between 1-10, 1-9, 1-8, 1-7, 1-6, 1-5, 1-4, 1-3, 1-2, 2-10, 2-9, 2-8, 2-7, 2-6,2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8,5-7, 5-6, 6-10, 6-9, 6-8, 6-7, 7-10, 7-9, 7-8, 8-10, 8-9, and 9-10. Such planes, according to some embodiments, exactly any of 1, 2, 3, 4, 5, 6, 7, 8, 9 or 10. In some embodiments, the planes of distributed tines (e.g., placed around the circumference of the cylinder), and be at an acute or oblique angle to axis 120 of the cylinder. The angle as well as the number of planes to which tines are arranged can be figured so as to provide a differing amount of retention force.

In some embodiments, the barb portions 118 of one set of tines arranged in one plane may lie opposite in direction then barb portions 118 of another set of tines (e.g., an adjacent set of tines) arranged on a second plane, so that when projecting outward, the two planes of tines restrict movement of the distal catheter 162 in both a distal and a proximal direction. In such embodiments, rotation of the catheter device in a first direction allows the barb portions to project outwards, and rotation of the catheter device in a second direction, opposite to the first (e.g., counter-clockwise opposite to clockwise), the tines fold or go downward (for example) so as to no longer engage or sufficiently engage tissue and allow removal of the catheter device.

In some embodiments, the device 150 can include one or more electrodes 164, and the device 150 can also include (in some embodiments) an electric pulse generator 160.

In some embodiments, a system for reversibly retaining at least one of a/the catheter 150 and a/the needle within tissue is provided and can include, the apparatus 100 (according to any of the above-noted embodiments and variations thereof), and, in some embodiments, at least one of, in some embodiments, at least two of, in some embodiments, at least three of, in some embodiments, at least four of, in some embodiments, at least five of, and in some embodiments, all of: a needle, an electrode 164, a liner extrusion, a jacket extrusion and a conductor wire 158. The liner extrusion can be, for example, 55D Pebax™ liner, which covers the jacket extrusion, which can be 55D Pebax™ jacket. The cylinder (with the tines) can ride on an additional liner, which can be 55D Pebax™ liner. The catheter 150 can include a first distal electrode, and a second more distal electrode of the electrodes 164. The conductor wire 158 can be used to, e.g., power the electrode(s).

In some embodiments, the apparatus 100 is spaced apart from a distal tip/end of the catheter (together the assembly) a predetermined distance which, in some embodiments, can be between 10 mm and 100 mm, and ranges therebetween. In some embodiments, the distance is preferably between 20 and 40 mm, and in some embodiments, between 25-35 mm, and in still further embodiments, approximately 30 mm. In some embodiments, electrodes included with the distal portion of the catheter, are placed distally to the retaining apparatus.

In some embodiments, a method for retaining at least one of a/the catheter 154, 162 and a/the needle within tissue is provided. In some such embodiments, the method includes providing the anchoring apparatus or system according to any of the above-noted embodiments, where:
the catheter 154, 162, cylinder 106, and optionally a/the needle (in some embodiments, the needle is first inserted into the proximal end of catheter 154 and is directed proximally through catheter 162. Accordingly, in some embodiments, at least a tip of the needle can project outward from the end of catheter 162), form an assembly,
the needle can be removably affixed to at least one of the catheter 154, 162 and cylinder 106
the needle can include a tip which can be electrified (e.g., in some embodiments, can be a part of the electrodes 164), and
the catheter 154, 162 can include the one or more electrodes 164 to apply an electric current to tissue;

In some embodiments, the needle is coated with Parylene-C, although in some embodiments, a small fraction on the proximal and distal ends is not coated with Parylene-C. The coating serves to insulate the needle, such that when an electric current is applied to the proximal end/tip of the needle, the tip is electrified. An insulation (not shown) can be included which is, inter alia, configured to electrically separate a needle from electrodes 164 of the device 150 (if included), once inserted into the catheter 154, 162. The needle (or assembly with the catheter) can be connected to an external nerve stimulator (not shown), which is connected via 156.

The method can also include one and/or another of: sterilizing a surface of tissue at an insertion site, anesthetizing the surface at the insertion site with a local anesthetic, and inserting the assembly through the surface of the insertion site towards a target nerve optionally under ultrasound imaging guidance. At least one of ultrasound images produced by the ultrasound imaging guidance and elicitation of an appropriate muscle twitch of a muscle proximate the target nerve via the electrified tip of the needle (in some embodiments), to confirm that the tip of the needle is adjacent the target nerve.

The method may further include unlocking or otherwise un-affixing the needle from the device 150, removing the needle from the tissue, rotating the cylinder 106 in a first direction such that the one or more tines 112 of the anchoring apparatus 100 deploy within the tissue, and rotating the cylinder 106 in a second direction opposite to the first direction such that the one or more tines 112 of the anchoring apparatus 100 wrap or otherwise fold to lie immediately adjacent the outer surface of the cylinder 106 and optionally received by a recess or opening 116 in at least one of the surface 110 of the cylinder and wall 108 of the cylinder 106.

In some embodiments, a method for use with a catheter pain management apparatus 150 or system is provided and includes providing the anchoring apparatus or anchoring system of any of the above-noted embodiments. In these embodiments, the catheter 102, cylinder 106, and optionally the needle, form an assembly, and the needle can be removably affixed to at least one of the catheter 154, 162 and cylinder 106. The needle includes a tip, and the catheter 154, 162 can include one or more electrodes 164 to apply an electric current to tissue. The method can further include, removably affixing the needle to the catheter, connecting the needle to an external nerve stimulator 160 which is configured to deliver a pulses of current at a predetermined amperage, frequency, and/or duration.

In some embodiments, the method may further include connecting tubing configured to be in continuity with a lumen of needle of device 150 to a syringe filled with a dextrose solution, flushing the lumen of the tubing and the lumen of the needle so as to remove any air therefrom, sterilizing a surface of tissue at an insertion site, anesthetizing the surface at the insertion site with a local anesthetic, injecting a dextrose solution through the needle to create an areas of fluid by hydro-dissection adjacent to the target nerve, and inserting the assembly through the surface of the insertion site towards a target nerve optionally under ultrasound imaging guidance. At least one of ultrasound images produced by the ultrasound imaging guidance and elicitation of an appropriate muscle twitch of a muscle proximate the target nerve via the one or more electrodes is configured to confirm that the tip of the needle is adjacent the target nerve.

The method may further include un-affixing the needle from the device 150, sliding the catheter 162 a predetermined distance into the hydro-dissected area adjacent the target nerve, removing the needle from the tissue, activating the pulse generator 160, optionally data linking the pulse generator to a smart device (not shown or other computer, e.g., remote or otherwise). The smart device can be configured to control the pulse generator 160 via at least one of a dedicated application operating on the smart device and a user interface thereof, which can establish at least one of a lower and upper stimulation threshold current for the pulse generator using at least one of the dedicated application and the user interface, rotating the cylinder 106 in a first direction such that the one or more tines 112 of the anchoring apparatus 100 deploy within the tissue so as to retain at least the catheter (e.g., 162) within the tissue, sealing the surface of the insertion site with a surgical glue, applying a waterproof dressing (not shown, but well known) configured to cover the catheter or portion of device 150 outside the surface of the tissue so as to keep an injection port/hub thereof outside the dressing, closing the injection port/hub with a clave, injecting a local anesthetic solution through a syringe to create a primary nerve block, capping the clave with a luer-lock component, and to remove the catheter, rotating the cylinder 106 in a second direction opposite to the first direction such that the one or more tines 112 of the anchoring apparatus 100 wrap or otherwise fold to lie immediately adjacent the outer surface 110 of the cylinder 106 and optionally received by a recess or opening 116 in at least one of the surface 110 of the cylinder 106 and wall 108 of the cylinder 106.

### Example Embodiments

Example 1: An anchoring apparatus for reversibly retaining a catheter or tube within tissue, the apparatus comprising a cylinder including a wall, an outer surface, and one or more tines, wherein each tine includes a portion thereof being at least one of flexibly integral and flexibly affixed to at least one of the outer surface and the wall, is configured to project away from the surface of the cylinder when in a deployed state, and in a undeployed state, is configured to wrap or otherwise fold upon rotation of the cylinder in a first direction so as to at least one of lie immediately adjacent the outer surface and/or be received by a recess or opening in at least one of the surface of the cylinder and wall of the cylinder.

Example 2: The apparatus of example 1, wherein the one or more tines comprise a plurality of tines.

Example 3: The apparatus of example 2, wherein the plurality of tines are arranged around the circumference of the cylinder.

Example 4: The apparatus of example 2, wherein the plurality of tines are equally spaced around the circumference of the cylinder.

Example 5: The apparatus of any of examples 1-4, wherein each tine is configured to be arranged within tissue.

Example 6: The apparatus of any of examples 1-4, wherein each tine includes a surface area configured to aid in retaining at least one of a catheter and needle within tissue.

Example 7: The apparatus of any of examples 1-6, wherein each tine includes at least one of a height, relative to the surface of the cylinder and an edge configured to interact with tissue so as to keep at least a distal end of a/the catheter and/or needle within the tissue upon an axial force being placed on the catheter and/or needle in a proximal direction thereof.

Example 8: The apparatus of any of examples 1-7, wherein each tine includes a barb portion configured to interact with tissue so as to keep the catheter and/or needle within the tissue and prevent longitudinal movement of the catheter at least in a first direction.

Example 9: The apparatus of any of examples 1-7, wherein each tine is configured as a propeller-like structure in the deployed state.

Example 10: The apparatus of example 9, wherein the propeller-like structure is configured with a pitch between 1 and 90 degrees relative to an axis of the cylinder.

Example 11: The apparatus of any of examples 1-10, wherein the one or more tines or plurality of tines comprise at least two tines.

Example 12: The apparatus of any of examples 1-10, wherein the one or more tines or plurality of tines comprise at least three tines.

Example 13: The apparatus of any of examples 1-10, wherein the one or more tines or plurality of tines comprise at least four tines.

Example 14: The apparatus of any of examples 1-10, wherein the one or more tines or plurality of tines are arranged in one or more planes lying at an angle to an/the longitudinal axis of the cylinder, wherein optionally, the angle is perpendicular the longitudinal axis.

Example 15: The apparatus of any of examples 1-10, wherein the one or more tines or plurality of tines are arranged in one or more planes arranged at an/the longitudinal axis of the cylinder.

Example 16: The apparatus of any of examples 1-10, wherein the one or more tines or plurality of tines are arranged in at least two planes arranged at an angle to the longitudinal axis of the cylinder.

Example 17: The apparatus of any of examples 1-16, wherein the catheter includes one or more electrodes.

Example 18: The apparatus of any of examples 1-17, wherein the catheter comprises a pulse generator.

Example 19: The apparatus of any of examples 1-18, wherein the one or more tines or plurality of tines are arranged in at least two planes lying at an angle to an/the longitudinal axis of the cylinder, wherein optionally, the angle is perpendicular the longitudinal axis.

Example 20: The apparatus of any of examples 1-18, wherein the one or more tines or plurality of tines are arranged in at least two planes lying at an angle to an/the longitudinal axis of the cylinder, optionally, the angle is perpendicular the longitudinal axis, a/the barb associated with the one or more or plurality of tines arranged on a first of the at least two planes is/are shaped and arranged so as to prevent longitudinal movement of the catheter in a first direction, and a/the barb associated with the one or more or plurality of tines arranged on a second of the at least two planes is/are shaped and arranged so as to prevent longitudinal movement of the catheter in a second direction opposite to the first direction.

Example 21: A system for reversibly retaining a catheter within tissue comprising the apparatus of any of examples 1-20, and at least one of: a catheter, a needle, an electrode, a liner extrusion, a jacket extrusion and a conductor wire.

Example 22: A system for reversibly retaining at least one of a catheter and needle within tissue comprising the apparatus of any of examples 1-20, and at least two of: a catheter, a needle, an electrode, a liner extrusion, a jacket extrusion and a conductor wire.

Example 23: A system for reversibly retaining at least one of a catheter and needle within tissue comprising the apparatus of any of examples 1-20 and at least three of: a catheter, a needle, an electrode, a liner extrusion, a jacket extrusion and a conductor wire.

Example 24: A system for reversibly retaining at least one of a catheter and needle within tissue comprising the apparatus of any of examples 1-20 and at least four of: a catheter, a needle, an electrode, a liner extrusion, a jacket extrusion and a conductor wire.

Example 25: A system for reversibly retaining at least one of a catheter and needle within tissue comprising the apparatus of any of examples 1-20 and at least five of: a catheter, a needle, an electrode, a liner extrusion, a jacket extrusion and a conductor wire.

Example 26: A system for reversibly retaining at least one of a catheter and needle within tissue comprising the apparatus of any of examples 1-20 and a catheter, a needle, an electrode, a liner extrusion, a jacket extrusion and a conductor wire.

Example 27: A method for retaining at least one of a catheter and a needle within tissue comprising providing the anchoring apparatus according to any of examples 1-20, or the anchoring system of any of examples 21-26, wherein the catheter and cylinder form an assembly, inserting the assembly through the surface of the insertion site towards a target area, where rotating the cylinder in a first direction such that the one or more tines of the anchoring apparatus deploy within the tissue, and rotating the cylinder in a second direction opposite to the first direction such that the one or more tines of the anchoring apparatus wrap and/or otherwise fold to lie immediately adjacent the outer surface of the cylinder and optionally are received by a recess or opening in at least one of the surface of the cylinder and wall of the cylinder.

Example 28: A method for retaining at least one of a catheter and a needle within tissue comprising providing the anchoring apparatus according to any of examples 1-20, or the anchoring system of any of examples 21-26, where the catheter and cylinder form an assembly, the catheter includes one or more electrodes to apply an electric current to tissue, sterilizing a surface of tissue at an insertion site, anesthetizing the surface at the insertion site with a local anesthetic, inserting the assembly through the surface of the insertion site towards a target nerve optionally under ultrasound imaging guidance, where at least one of ultrasound images produced by the ultrasound imaging guidance and elicitation of an appropriate muscle twitch of a muscle proximate the target nerve via the one or more electrodes is configured to confirm that a portion of catheter is adjacent the target nerve, rotating the cylinder in a first direction such that the one or more tines of the anchoring apparatus deploy within the tissue, and rotating the cylinder in a second direction opposite to the first direction such that the one or more tines of the anchoring apparatus wrap and/or otherwise fold to lie immediately adjacent the outer surface of the cylinder and optionally are received by a recess or opening in at least one of the surface of the cylinder and wall of the cylinder.

Example 29: A method for use of a catheter pain management apparatus or system comprising providing the anchoring apparatus according to any of examples 1-20, or the anchoring system of any of examples 21-26 where the catheter and cylinder, and optionally a needle, form an assembly, the catheter includes a portion including a medical device for positioning and/or delivering the medical device adjacent a particular area of tissue, removably affixing the needle to the catheter, connecting the needle to a pulse generator configured to deliver a pulses of current at a predetermined amperage, frequency, and/or duration, connecting tubing configured to be in continuity with a lumen of the needle to a syringe filled with a dextrose solution, flushing the lumen of the tubing and needle so as to remove any air therefrom, sterilizing a surface of tissue at an insertion site, anesthetizing the surface at the insertion site with a local anesthetic, injecting a dextrose solution through the needle to create an areas of fluid by hydro-dissection adjacent to the target nerve, inserting the assembly through the surface of the insertion site towards a target nerve optionally under ultrasound imaging guidance, where at least one of ultrasound images produced by the ultrasound imaging guidance and elicitation of an appropriate muscle twitch of a muscle proximate the target nerve via the one or more electrodes is configured to confirm that the tip of the needle is adjacent the target nerve, un-affixing the needle from the catheter, sliding the catheter a predetermined distance into the hydro-dissected area adjacent the target nerve, removing the needle from the tissue, activating the pulse generator, optionally data linking the pulse generator to a smart device, wherein the smart device is configured to control the pulse generator via at least one of a dedicated application operating on the smart device and a user interface thereof, establishing at least one of a lower and upper stimulation threshold current for the pulse generator using at least one of the dedicated application and user interface, rotating the cylinder in a first direction such that the one or more tines of the anchoring apparatus deploy within the tissue so as to retain at least the catheter within the tissue, sealing the surface of the insertion site with a surgical glue, applying a waterproof dressing configured to cover the catheter outside the surface of the tissue so as to keep an injection port/hub thereof outside the dressing, closing the injection port/hub with a clave, injecting a local anesthetic solution through a syringe to create a primary nerve block, capping the clave with a luer-lock component, and to remove the catheter, rotating the cylinder in a second direction opposite to the first direction such that the one or more tines of the anchoring apparatus wrap or otherwise fold to lie immediately adjacent the outer surface of the cylinder and optionally received by a recess or opening in at least one of the surface of the cylinder and wall of the cylinder.

### General Considerations

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that any and all parameters, dimensions, materials, and configurations described herein are meant to be an example and that the actual parameters, dimensions, materials, and/or configurations will depend upon the specific application or applications for which the inventive teachings disclosed herein is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the claims supported by the disclosure, and equivalents thereto, inventive embodiments may be practiced otherwise than as specifically described and claimed. Inventive embodiments of the present disclosure are also directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to such embodiments are distinguishable from the prior art by including one or more negative limitations.

Also, various inventive concepts may be embodied as one or more methods. The acts performed as part of the method may be ordered in any suitable way. Accordingly, embodiments may be constructed in which acts are performed in an order different than illustrated, which may include performing some acts simultaneously.

The indefinite articles “a” and “an,” as used herein in the specification and in the claims, unless clearly indicated to the contrary, should be understood to mean “at least one.” The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. What is currently claimed:

1. An anchoring apparatus for reversibly retaining a catheter or tube within tissue, the apparatus comprising a cylinder including a wall, an outer surface, and one or more tines, wherein:
each tine:
includes a portion thereof being at least one of flexibly integral and flexibly affixed to at least one of the outer surface and the wall;
is configured to project away from the surface of the cylinder when in a deployed state;
and
in a undeployed state, is configured to wrap or otherwise fold upon rotation of the cylinder in a first direction so as to at least one of lie immediately adjacent the outer surface and/or be received by a recess or opening in at least one of the surface of the cylinder and wall of the cylinder.

2. The apparatus of claim 1, wherein the one or more tines comprise a plurality of tines.

3. The apparatus of claim 2, wherein the plurality of tines are arranged around the circumference of the cylinder.

4. The apparatus of claim 2, wherein the plurality of tines are equally spaced around the circumference of the cylinder.

5. The apparatus of claim 1, wherein each tine is configured to be arranged within tissue.

6. The apparatus of claim 1, wherein each tine includes a surface area configured to aid in retaining at least one of a catheter and a needle within tissue.

7. The apparatus of claim 1, wherein each tine includes at least one of a height, relative to the surface of the cylinder and an edge configured to interact with tissue so as to keep at least a distal end of a/the catheter and/or a needle within the tissue upon an axial force being placed on the catheter and/or a needle in a proximal direction thereof.

8. The apparatus of claim 1, wherein each tine includes a barb portion configured to interact with tissue so as to keep the catheter and/or a needle within the tissue and prevent longitudinal movement of the catheter at least in a first direction.

9. The apparatus of any of claim 1, wherein each tine is configured as a propeller-like structure in the deployed state.

10. The apparatus of claim 9, wherein the propeller-like structure is configured with a pitch between 1 and 90 degrees relative to an axis of the cylinder.

11. The apparatus of claim 1, wherein the one or more tines or plurality of tines comprise at least two tines.

12. The apparatus of claim 1, wherein the one or more tines or plurality of tines comprise at least three tines.

13. The apparatus of claim 1, wherein the one or more tines or plurality of tines comprise at least four tines.

14. The apparatus of claim 1, wherein the one or more tines or plurality of tines are arranged in one or more planes lying at an angle to an/the longitudinal axis of the cylinder, wherein optionally, the angle is perpendicular the longitudinal axis.

15. The apparatus of claim 1, wherein the one or more tines or plurality of tines are arranged in one or more planes arranged at an/the longitudinal axis of the cylinder.

16. The apparatus of claim 1, wherein the one or more tines or plurality of tines are arranged in at least two planes arranged at an angle to the longitudinal axis of the cylinder.

17. The apparatus of claim 1, wherein the catheter includes one or more electrodes.

18. The apparatus of claim 1, wherein the catheter comprises a pulse generator.

19. The apparatus of claim 1, wherein the one or more tines or plurality of tines are arranged in at least two planes lying at an angle to an/the longitudinal axis of the cylinder, wherein optionally, the angle is perpendicular the longitudinal axis.

20. The apparatus of claim 1, wherein:
the one or more tines or plurality of tines are arranged in at least two planes lying at an angle to an/the longitudinal axis of the cylinder,
optionally, the angle is perpendicular the longitudinal axis,
a/the barb associated with the one or more or plurality of tines arranged on a first of the at least two planes is/are shaped and arranged so as to prevent longitudinal movement of the catheter in a first direction, and
a/the barb associated with the one or more or plurality of tines arranged on a second of the at least two planes is/are shaped and arranged so as to prevent longitudinal movement of the catheter in a second direction opposite to the first direction.

21. A system for reversibly retaining a catheter within tissue comprising:
the apparatus of any of claims 1-20;
and
at least one of: a catheter, a needle, an electrode, a liner extrusion, a jacket extrusion and a conductor wire.

22. A system for reversibly retaining a catheter within tissue comprising:
the apparatus of any of claims 1-20;
and
at least two of: a catheter, a needle, an electrode, a liner extrusion, a jacket extrusion and a conductor wire.

23. A system for reversibly retaining a catheter within tissue comprising:
the apparatus of any of claims 1-20;
and
at least three of: a catheter, a needle, an electrode, a liner extrusion, a jacket extrusion and a conductor wire.

24. A system for reversibly retaining a catheter within tissue comprising:
the apparatus of any of claims 1-20;
and
at least four of: a catheter, a needle, an electrode, a liner extrusion, a jacket extrusion and a conductor wire.

25. A system for reversibly retaining at least one of a catheter and needle within tissue comprising:
the apparatus of any of claims 1-20;
and
at least five of: a catheter, a needle, an electrode, a liner extrusion, a jacket extrusion and a conductor wire.

26. A system for reversibly retaining at least one of a catheter and needle within tissue comprising:
the apparatus of any of claims 1-20;
and
a catheter, a needle, an electrode, a liner extrusion, a jacket extrusion and a conductor wire.

27. A method for retaining a catheter within tissue comprising:
providing the anchoring apparatus according to any of claims 1-20, or the anchoring system of any of claims 21-26, wherein the catheter and cylinder form an assembly,
inserting the assembly through the surface of the insertion site towards a target area, wherein:
rotating the cylinder in a first direction such that the one or more tines of the anchoring apparatus deploy within the tissue;
and
rotating the cylinder in a second direction opposite to the first direction such that the one or more tines of the anchoring apparatus wrap and/or otherwise fold to lie immediately adjacent the outer surface of the cylinder and optionally are received by a recess or opening in at least one of the surface of the cylinder and wall of the cylinder.

28. A method for retaining at least one of a catheter and a needle within tissue comprising:
providing the anchoring apparatus according to any of claims 1-20, or the anchoring system of any of claims 21-26, wherein:
the catheter and cylinder form an assembly,
the catheter includes one or more electrodes to apply an electric current to tissue;
sterilizing a surface of tissue at an insertion site;
anesthetizing the surface at the insertion site with a local anesthetic,
inserting the assembly through the surface of the insertion site towards a target nerve optionally under ultrasound imaging guidance, wherein:
at least one of ultrasound images produced by the ultrasound imaging guidance and elicitation of an appropriate muscle twitch of a muscle proximate the target nerve via the one or more electrodes is configured to confirm that a portion of catheter is adjacent the target nerve;
rotating the cylinder in a first direction such that the one or more tines of the anchoring apparatus deploy within the tissue;
and
rotating the cylinder in a second direction opposite to the first direction such that the one or more tines of the anchoring apparatus wrap and/or otherwise fold to lie immediately adjacent the outer surface of the cylinder and optionally are received by a recess or opening in at least one of the surface of the cylinder and wall of the cylinder.

29. A method for use of a catheter pain management apparatus or system comprising:
providing the anchoring apparatus according to any of claims 1-20, or the anchoring system of any of claims 21-26 wherein:
the catheter and cylinder, and optionally a needle, form an assembly,
the catheter includes a portion including a medical device for positioning and/or delivering the medical device adjacent a particular area of tissue,
removably affixing the needle to the catheter;
connecting the needle to a pulse generator configured to deliver a pulses of current at a predetermined amperage, frequency, and/or duration;
connecting tubing configured to be in continuity with a lumen of the needle to a syringe filled with a dextrose solution;
flushing the lumen of the tubing and needle so as to remove any air therefrom;
sterilizing a surface of tissue at an insertion site;
anesthetizing the surface at the insertion site with a local anesthetic,
injecting a dextrose solution through the needle to create an areas of fluid by hydro-dissection adjacent to the target nerve;
inserting the assembly through the surface of the insertion site towards a target nerve optionally under ultrasound imaging guidance, wherein:
at least one of ultrasound images produced by the ultrasound imaging guidance and elicitation of an appropriate muscle twitch of a muscle proximate the target nerve via the one or more electrodes is configured to confirm that the tip of the needle is adjacent the target nerve;
un-affixing the needle from the catheter;
sliding the catheter a predetermined distance into the hydro-dissected area adjacent the target nerve;
removing the needle from the tissue;
activating the pulse generator;
optionally data linking the pulse generator to a smart device, wherein the smart device is configured to control the pulse generator via at least one of a dedicated application operating on the smart device and a user interface thereof;
establishing at least one of a lower and upper stimulation threshold current for the pulse generator using at least one of the dedicated application and user interface;
rotating the cylinder in a first direction such that the one or more tines of the anchoring apparatus deploy within the tissue so as to retain at least the catheter within the tissue;
sealing the surface of the insertion site with a surgical glue;
applying a waterproof dressing configured to cover the catheter outside the surface of the tissue so as to keep an injection port/hub thereof outside the dressing;
closing the injection port/hub with a clave;
injecting a local anesthetic solution through a syringe to create a primary nerve block;
capping the clave with a luer-lock component;
and
to remove the catheter, rotating the cylinder in a second direction opposite to the first direction such that the one or more tines of the anchoring apparatus wrap or otherwise fold to lie immediately adjacent the outer surface of the cylinder and optionally received by a recess or opening in at least one of the surface of the cylinder and wall of the cylinder.

30. A method, system, apparatus or device according to any one or more of the embodiments disclosed herein.
